SEQUENCE LISTING (10 sequences)

SEQ ID NO: 1 - DNA, 44 bp, Artificial Sequence
  Description of Artificial Sequence Synthetic oligonucleotide
ccatctcatccctgcgtgtcccatctgttccctccctgtctcag

SEQ ID NO: 2 - DNA, 12 bp, Artificial Sequence
  Description of Artificial Sequence Synthetic oligonucleotide
ctgagacaggga

SEQ ID NO: 3 - DNA, 44 bp, Artificial Sequence
  Description of Artificial Sequence Synthetic oligonucleotide
cctatcccctgtgtgccttgcctatcccctgttgcgtgtctcag

SEQ ID NO: 4 - DNA, 12 bp, Artificial Sequence
  Description of Artificial Sequence Synthetic oligonucleotide
ctgagacacgca

SEQ ID NO: 5 - DNA, 24 bp, Artificial Sequence
  Description of Artificial Sequence Synthetic primer
atctcatccctgcgtgtcccatct

SEQ ID NO: 6 - DNA, 24 bp, Artificial Sequence
  Description of Artificial Sequence Synthetic primer
tatcccctgtgtgccttgcctatc

SEQ ID NO: 7 - DNA, 45 bp, Artificial Sequence
  Description of Artificial Sequence Synthetic oligonucleotide
ccatctcatccctgcgtgtcccatctgttccctccctgtctcagc

SEQ ID NO: 8 - DNA, 45 bp, Artificial Sequence
  Description of Artificial Sequence Synthetic oligonucleotide
cctatcccctgtgtgccttgcctatcccctgttgcgtgtctcagc

SEQ ID NO: 9 - DNA, 25 bp, Artificial Sequence
  Description of Artificial Sequence Synthetic oligonucleotide
atctcatccctgcgtgtcccatctc

SEQ ID NO: 10 - DNA, 25 bp, Artificial Sequence
  Description of Artificial Sequence Synthetic oligonucleotide
tatcccctgtgtgccttgcctatcc

(19) United States
(12) Patent Application Publication
(10) Pub. No.: US 2010/0167952 A1
(43) Pub. Date: Jul. 1, 2010

(54) SUPPRESSION OF SECONDARY CAPTURE IN MICROARRAY ASSAYS

(76) Inventors: Thomas Albert, Madison, WI (US); Jeff Jeddeloh, Verona, WI (US); Bradley Swanson, Madison, WI (US)

Correspondence Address: ROCHE DIAGNOSTICS OPERATIONS INC., 9115 Hague Road, Indianapolis, IN, 46250-0457, US

(21) Appl. No.: 12/553,251

(22) Filed: Sep. 3, 2009

Related U.S. Application Data
(60) Provisional application No. 61/111,881, filed on Nov. 6, 2008.

(51) Int. Cl. C07H 21/04; C40B 40/06

(52) U.S. Cl. 506/16; 536/24.2

(57) ABSTRACT

The present invention provides for compositions, methods and systems for targeted sequence enrichment. In particular, the present invention provides for enriching for targeted nucleic acid sequences during hybridizations in microarray assays by suppressing secondary capture of non-target nucleic acid sequences.

### RELATED APPLICATIONS

This application claims priority to U.S. Provisional Application No. 61/111,881 filed Nov. 6, 2008.

### FIELD OF THE INVENTION

The present invention provides for compositions, methods and systems for targeted sequence enrichment. In particular, the present invention provides for enriching for targeted nucleic acid sequences during hybridizations in microarray assays by suppressing secondary capture of non-target nucleic acid sequences.

### BACKGROUND OF THE INVENTION

The advent of nucleic acid microarray technology makes it possible to build an array of millions of nucleic acid sequences in a very small area, for example on a microscope slide (e.g., U.S. Pat. Nos. 6,375,903 and 5,143,854). Initially, such arrays were created by spotting pre-synthesized DNA sequences onto slides. However, the construction of maskless array synthesizers (MAS) as described in U.S. Pat. No. 6,375,903 now allows for the in situ synthesis of oligonucleotide sequences directly on the slide itself.

Using a MAS instrument, the selection of oligonucleotide sequences to be constructed on the microarray is under software control such that it is now possible to create individually customized arrays based on the particular needs of an investigator. In general, MAS-based oligonucleotide microarray synthesis technology allows for the parallel synthesis of millions of unique oligonucleotide features in a very small area of a standard microscope slide. With the availability of the entire genomes of hundreds of organisms, for which a reference sequence has generally been deposited into a public database, microarrays have been used to perform sequence analysis on nucleic acids isolated from a myriad of organisms.

Nucleic acid microarray technology has been applied to many areas of research and diagnostics, such as gene expression and discovery, mutation detection, allelic and evolutionary sequence comparison, genome mapping, drug discovery, and more. Many applications require searching for genetic variants and mutations across the entire human genome that underlies human diseases. In the case of complex diseases, these searches generally result in a single nucleotide polymorphism (SNP) or set of SNPs associated with diseases and/or disease risk. Identifying such SNPs has proved to be an arduous and frequently fruitless task because resequencing large regions of genomic DNA, usually greater than 100 kilobases (Kb), from affected individuals or tissue samples is required to find a single base change or to identify all sequence variants. Other applications involve the identification of gains and losses of chromosomal sequences which may also be associated with cancer, such as lymphoma (Martinez-Climent J A et al., 2003, Blood 101:3109-3117), gastric cancer (Weiss. M M et al., 2004, Cell. Oncol. 26:307-317), breast cancer (Callagy G et al., 2005, J. Path. 205: 388-396) and prostate cancer (Paris, P L et al., 2004, Hum. Mol. Gen. 13:1303-1313). As such, microarray technology is a tremendously useful tool for scientific investigators and clinicians in their understanding of diseases and therapeutic regimen efficacy in treating diseases.

The genome is typically complex to be studied as a whole, and techniques must be used to reduce the complexity of the genome. To address this problem, one solution is to reduce certain types of abundant sequences from a DNA sample, as found in U.S. Pat. No. 6,013,440. Alternatives employ methods and compositions for enriching genomic sequences as described, for example, in Albert et al. (2007, Nat. Meth., 4:903-5), Okou et al. (2007, Nat. Meth. 4:907-9), Olson M. (2007, Nat. Meth. 4:891-892), Hodges et al. (2007, Nat. Genet. 39:1522-1527) and as found in U.S. patent application Ser. Nos. 11/638,004, 11/970,949, and 61/032,594. Albert et al. disclose an alternative that is both cost-effective and rapid in effectively reducing the complexity of a genomic sample in a user defined way to allow for further processing, and analysis. Lovett ct al. (1991, Proc. Natl. Acad. Sci. 88:9628-9632) also describes a method for genomic selection using bacterial artificial chromosomes. Reducing the complexity of a genome by practicing target sequence enrichment followed by sequencing is far superior to measuring hybridization events alone. Hybridization events allow the hybridization of any species in a microarray; both target sequences and non-target sequences alike. By practicing complexity reduction and sequence enrichment, an investigator increases the on-target sequences captured (e.g., those sequences that are the focus of the assay) while decreasing the amount of non-target sequences captured (e.g., those not the focus of the assay).

However, an issue associated with any microarray assay is the event of cross capture of repetitive nucleic acid sequences, also known as secondary capture, of non-target nucleic acid sequences on the array during hybridization of the target nucleic acids. Secondary capture decreases the efficiency of complexity reduction and other microarray assays, in effect potentially swamping out the desired target capture by non-target capture leading to decreased target capture efficiency.

As such, what are needed are methods for suppressing secondary capture reactions from occurring on a microarray assay thereby increasing the efficiency of target nucleic acid capture for investigative endeavors.

### SUMMARY OF THE INVENTION

Secondary capture reactions on a microarray format lead to decreased efficiency in capturing target nucleic acids. This decreased efficiency is seen in the percent of on-target reads resulting from a microarray assay, such that when secondary capture is not suppressed the amount of non-target nucleic acids captured increases and the target nucleic acids decrease. The present invention is summarized as methods, systems and compositions for suppressing secondary capture in a microarray assay. Certain illustrative embodiments of the invention are described below. The present invention is not limited to these embodiments.

Embodiments of the present invention comprise immobilized nucleic acid probes to capture target nucleic acid sequences from, for example, a genomic sample by hybridizing the sample to probes, or probe derived amplicons, on a solid support or in solution. Hybridization reactions as described herein comprise the addition of species specific blocking DNA to the reactions in a microarray assay. Species specific blocking DNA includes, for example, the incorporation of human C₀t-1 DNA with human target nucleic acids during human specific microarray hybridizations, the incorporation of mouse C₀t-1 DNA with mouse target nucleic acids during mouse specific microarray hybridizations, and the incorporation of maize C₀t-1 DNA with maize target nucleic acid during maize specific microarray hybridizations.

Further embodiments of the present invention comprise immobilized nucleic acid probes to capture target nucleic acid sequences from, for example, a genomic sample by hybridizing the sample to probes, or probe derived amplicons, on a solid support or in solution, wherein the target nucleic acid is affixed with adapter linkers on one or both of the 5′ and 3′ ends of a fragmented nucleic acid sample, adapter linkers being useful for ligation mediated polymerase chain reaction (LM-PCR) methods and for sequencing applications. Hybridization reactions as described herein comprise the addition of species specific blocking DNA as previously described and/or the incorporation of a synthetic hybridization blocking oligonucleotide to hybridization reactions on a microarray. Species specific blocking DNA has been previously described. Hybridization blocking oligonucleotides are incorporated in microarray hybridizations to block secondary capture due to, for example, adapter mediated secondary capture.

The incorporation of species specific blocking DNA and/or hybridization blocking oligonucleotides in complexity reduction assays as provided by the present invention suppresses secondary capture thereby increasing the amount of on target nucleic acid sequences (e.g., the desired target sequences) captured during hybridization when compared to using non-species specific blocking DNA during hybridizations. The captured target nucleic acids arc preferably washed and eluted off of the probes. In some embodiments, the present invention provides for the enrichment of targeted sequences and suppression of secondary capture for non-targeted sequences, in a solution based format. It is contemplated that the present invention is not limited to the microarray substrate. Microarray substrates include, but are not limited to, slide, chip, beads, solution based, tube, column, wells, plates, and the like.

Genomic samples are used herein for descriptive purposes, but it is understood that other non-genomic samples could be subjected to the same procedures as the present invention provides for the suppression of secondary non-target capture in conjunction with any nucleic acid target regardless of origin. Increases in efficiency of target enrichment provided by the present invention offer investigators superior tools for use in research and therapeutics associated with disease and disease states such as cancers (Durkin et al., 2008, Proc. Natl. Acad. Sci. 105:246-251; Natrajan et al., 2007, Genes, Chr. And Cancer 46:607-615; Kim et al., 2006, Cell 125:1269-1281; Stallings et al., 2006 Can. Res. 66:3673-3680), genetic disorders (Balciuniene et al., Am. J. Hum. Genet. In press), mental diseases (Walsh et al., 2008, Science 320:539-543; Roohi et al., 2008, J. Med. Genet. Epub 18 Mar. 2008; Sharp et al., 2008, Nat. Genet. 40:322-328; Kumar et al., 2008, Hum. Mol. Genet. 17:628-638) and evolutionary and basic research (Lee et al., 2008, Hum. Mol. Gen. 17:1127-1136; Jones et al., 2007, BMC Genomics 8:402; Egan et al., 2007, Nat. Genet. 39:1384-1389; Levy et al., 2007, PLoS Biol. 5:e254; Ballif et al., 2007, Nat. Genet. 39:1071-1073; Scherer et al., 2007, Nat. Genet. S7-S15; Feuk et al., 2006, Nat. Rev. Genet. 7:85-97), to name a few.

The present invention provides methods of isolating and reducing the genetic complexity of a plurality of nucleic acid molecules, the method comprising the steps of exposing fragmented, denatured nucleic acid molecules of said population to the same or multiple, different oligonucleotide probes that are bound on a solid support under hybridizing conditions to capture nucleic acid molecules that specifically hybridize to said probes, or exposing fragmented, denatured nucleic acid molecules of said population to the same or multiple, different oligonucleotide probes under hybridizing conditions followed by binding the complexes of hybridized molecules to a solid support to capture nucleic acid molecules that specifically hybridize to said probes, wherein in both cases said fragmented, denatured nucleic acid molecules have an average size of about 100 to about 1000 nucleotide residues, preferably about 250 to about 800 nucleotide residues and most preferably about 400 to about 600 nucleotide residues, separating unbound and non-specifically hybridized nucleic acids from the captured molecules, eluting the captured molecules, and optionally repeating the aforementioned processes for at least one further cycle with the eluted captured molecules and/or sequencing the enriched target nucleic acids.

In some embodiments, the target nucleic acid molecules are selected from an animal, a plant or a microorganism. If only limited samples of nucleic acid are available, the nucleic acids may be amplified, for example by whole genome amplification, prior to practicing the methods of the present invention. Prior amplification may be necessary for performing the inventive method(s), for example, for forensic purposes (e.g. in forensic medicine for genetic identity purposes).

In some embodiments, the population of target nucleic acid molecules is a population of genomic DNA molecules. In such embodiments, probes are selected from one or a plurality of sequences that; for example, define one or a plurality of exons, introns or regulatory sequences from a plurality of genetic loci, or a plurality of probes that define the complete sequence of at least one single genetic locus, said locus having a size of at least 100 kb, preferably at least 1 Mb, or at least one of the sizes as specified above, one or a plurality of probes that define single nucleotide polymorphisms (SNPs), or a plurality of probes that define an array, for example a tiling array designed to capture the complete sequence of at least one complete chromosome.

In some embodiments, the present invention comprises the step of ligating adapter molecules to one or both, preferably both ends of the nucleic acid molecules prior to or after exposing fragmented nucleic samples to the probes for hybridization. In some embodiments, methods of the present invention further comprise the amplifying of the target nucleic acid molecules with at least one primer, said primer comprising a sequence which specifically hybridizes to the sequence of said adapter molecule(s). In some embodiments, the adapter molecules are self-complementary, non-complementary, or are Y-adapters (e.g., oligonucleotides that, once annealed, comprise a complementary end and a non-complementary end, the complementary end of which is annealed to fragmented nucleic acid samples). In some embodiments, the amplified target nucleic acid sequences may be sequenced, hybridized to a resequencing or SNP-calling array and the sequence or genotypes may be further analyzed.

In some embodiments, the present invention provides a complexity reduction method for target nucleic acid sequences in a genomic sample, such as exons or variants, preferably SNP sites. This can be accomplished by synthesizing one or more genomic probes specific for a region of the genome to capture complementary target nucleic acid sequences contained in a complex genomic sample. The enrichment methods comprise the inclusion of one or both of species specific blocking DNA and hybridization blocking oligonucleotides.

In some embodiments, the present invention further comprises determining the nucleic acid sequence of the enriched and eluted target molecules, in particular by means of performing sequencing reactions.

In some embodiments, the present invention is directed to a kit comprising compositions and reagents for performing a method according to the present invention. Such a kit may comprise, but is not limited to, a double stranded adapter molecule, a solid support comprising a plurality of hybridization probes for any particular microarray application (e.g., comparative genomic hybridization, expression, chromatin immunoprecipitation, comparative genomic sequencing, etc.) and one or more of a species specific blocking DNA and a hybridization blocking oligonucleotide. In some embodiments, a kit comprises two different double stranded adapter molecules. A kit may further comprise at least one or more other components selected from DNA polymerase, T4 polynucleotide kinase, T4 DNA ligase, hybridization solution(s), wash solution(s), and/or elution solution(s).

### DESCRIPTION OF FIGURES

FIG. 1 exemplifies two different types of secondary capture.

FIG. 2 is exemplary of the effect of different types of blocking DNAs in a microarray with human target DNA. Human represents human C₀t-1, salmon represents salmon sperm DNA, mouse represents mouse C₀t-1 and none is no blocking DNA (negative control).

FIG. 3 exemplifies the importance of utilizing species specific blocking DNA in microarray capture experiments.

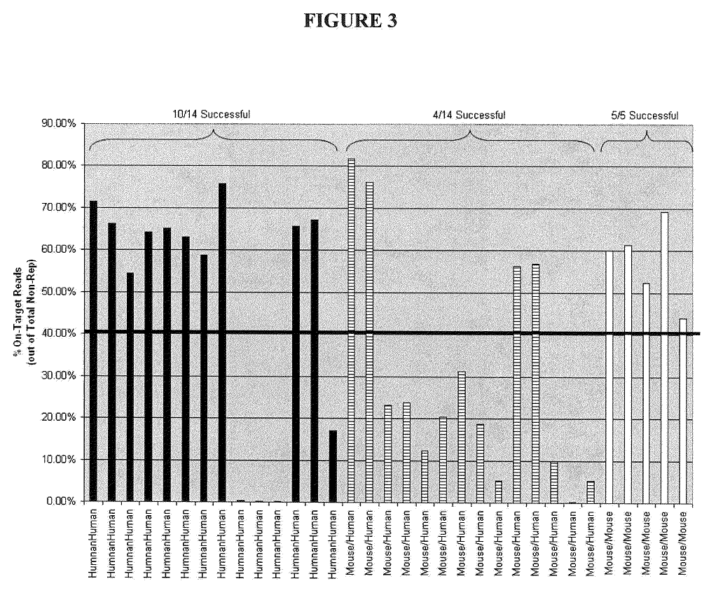

FIG. 4 shows an exemplary workflow for one method of performing sequence capture of targeted sample nucleic acids. In this exemplary workflow, a DNA sample is fragmented and adapters (e.g., linkers) are added to the ends of the fragmented DNA, for example using a kit for creating a DNA library. Single stranded templates are amplified using the linkers as affixed to the ends of the fragmented DNA, for example by ligation mediated polymerase chain reaction methods (LM-PCR). The samples are denatured, hybridized to a microarray substrate, washed and eluted. The eluted samples are amplified using the adapter sequences and subsequently sequenced.

FIG. 5 exemplifies the importance of utilizing hybridization blocking oligonucleotides in a microarray capture assay to block adapter mediated secondary hybridization when adapters are ligated to target nucleic acids.

### DEFINITIONS

As used herein, the term “sample” is used in its broadest sense. In one sense, it is meant to include a specimen or culture obtained from any source, preferentially a biological source, both eukaryotic or prokaryotic. Biological samples may be obtained from animals (including humans) and encompass fluids, solids, and tissues. Biological samples include blood products, such as plasma, serum and the like. A sample from a non-human animal includes, but is not limited to, a biological sample from vertebrates such as rodents, non-human primates, ovines, bovines, ruminants, lagomorphs, porcines, caprines, equines, canines, felines, ayes, etc. Further, a sample as used herein includes biological samples from plants, for example a sample derived from any organism as found in the kingdom Plantae (e.g., monocot, dicot, etc.). A sample can also be from fungi, algae, bacteria, and the like. It is contemplated that the present invention is not limited to the origin of the sample. A sample as used herein is typically, a “sample of nucleic acids” or a “nucleic acid sample”, or a “target nucleic acid sample”, or a “target sample” comprising nucleic acids (e.g., DNA, RNA, cDNA, mRNA, tRNA, miRNA, etc.) from any source. As such, a nucleic acid sample used in methods and systems of the present invention is a nucleic acid sample derived from any organism, either eukaryotic or prokaryotic.

As used herein, the term “target nucleic acid molecules” and “target nucleic acid sequences” are used interchangeably and refer to molecules or sequences from a target genomic region to be studied. The pre-selected probes determine the range of targeted nucleic acid molecules. Thus, the “target” is sought to be sorted out from other nucleic acid sequences. A “segment” is defined as a region of nucleic acid within the target sequence, as is a “fragment” or a “portion” of a nucleic acid sequence. As such, “on-target reads” are the percentage or number of target nucleic acids that are sequenced and found to be the sequences desired by an investigator.

As used herein, the term “isolate” when used in relation to a nucleic acid, as in “isolating a nucleic acid” refers to a nucleic acid sequence that is identified and separated from at least one component or contaminant with which it is ordinarily associated in its natural source. Isolated nucleic acid is in a form or setting that is different from that in which it is found in nature. In contrast, non-isolated nucleic acids as nucleic acids such as DNA and RNA found in the state they exist in nature. The isolated nucleic acid, oligonucleotide, or polynucleotide may be present in single-stranded or double-stranded form.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the term “hybridization” is used in reference to the pairing of complementary nucleic acids. Hybridization and the strength of hybridization (e.g., the strength of the association between the nucleic acids) is affected by such factors as the degree of complementary between the nucleic acids, stringency of the conditions involved, the melting temperature (Tₘ) of the formed hybrid, and the G:C ratio of the nucleic acids. While the invention is not limited to a particular set of hybridization conditions, stringent hybridization conditions are preferably employed. Stringent hybridization conditions are sequence dependent and differ with varying environmental parameters (e.g., salt concentrations, presence of organics, etc.). Generally; “stringent” conditions are selected to be about 50° C. to about 20° C. lower than the Tₘ for the specific nucleic acid sequence at a defined ionic strength and pH. Preferably, stringent conditions are about 5° C. to 10° C. lower than the thermal melting point for a specific nucleic acid bound to a complementary nucleic acid. The Tₘ is the temperature (under defined ionic strength and pH) at which 50% of a nucleic acid (e.g., target nucleic acid) hybridizes to a perfectly matched probe.

“Stringent conditions” or “high stringency conditions,” for example, can be hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 mg/ml), 0.1% SDS, and 10% dextran sulfate at 42° C., with washes at 42° C. in 0.2% SSC (sodium chloride/sodium citrate) and 50% formamide at 55° C., followed by a wash with 0.1×SSC containing EDTA at 55° C. By way of example, but not limitation, it is contemplated that buffers containing 35% formamide, 5×SSC, and 0.1% (w/v) sodium dodecyl sulfate (SDS) are suitable for hybridizing under moderately non-stringent conditions at 45° C. for 16-72 hours.

Furthermore, it is envisioned that the formamide concentration may be suitably adjusted between a range of 20-45% depending on the probe length and the level of stringency desired. Additional examples of hybridization conditions are provided in several sources, including Molecular Cloning: A Laboratory Manual, Eds. Sambrook et al., Cold Spring Harbour Press (incorporated herein by reference in its entirety).

Similarly, “stringent” wash conditions are ordinarily determined empirically for hybridization of a target to a probe, or in the present invention, a probe derived amplicon. The amplicon/target are hybridized (for example, under stringent hybridization conditions) and then washed with buffers containing successively lower concentrations of salts, or higher concentrations of detergents, or at increasing temperatures until the signal-to-noise ratio for specific to non-specific hybridization is high enough to facilitate detection of specific hybridization. Stringent temperature conditions will usually include temperatures in excess of about 30° C., more usually in excess of about 37° C., and occasionally in excess of about 45° C. Stringent salt conditions will ordinarily be less than about 1000 mM, usually less than about 500 mM, more usually less than about 150 mM (Wetmur et al., 1966, J. Mol. Biol., 31:349-370; Wetmur, 1991, Critical Reviews in Biochemistry and Molecular Biology, 26:227-259, incorporated by reference herein in their entireties).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as DNA polymerase and at a suitable temperature and pH). The primer is preferably single stranded for maximum efficiency in amplification. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (e.g., a sequence of nucleotides), whether occurring naturally as in a purified restriction digest or produced synthetically, recombinantly or by PCR amplification, that is capable of hybridizing to at least a portion of another oligonucleotide of interest, for example target nucleic acid sequences. A probe may be single-stranded or double-stranded. Probes are useful in the detection, identification and isolation of particular gene sequences. A probe as used herein is typically affixed to a microarray substrate, either by in situ synthesis using MAS or by any other method known to a skilled artisan, and hybridizes to a target nucleic acid.

As used herein, the term “adapter” (or “adaptor”) is a double stranded oligonucleotide of defined (or known) sequence which is affixed to one or both ends of sample DNA molecules. Sample DNA molecules may be fragmented or not before their addition. In the case where adapters are added to both ends of the sample DNA molecule, the adapters may be the same (i.e homologous sequence on both ends) or different (i.e heterologous sequences at each end). For the purposes of ligation-mediated polymerase chain reaction (LM-PCR), the terms “adapter” and “linker” are used interchangeably. The two strands of the adapter may be self-complementary, non-complementary or partially complementary (e.g. Y-shaped). Adapters typically range from 12 nucleotide residues to 100 nucleotide residues, preferably from 18 nucleotide residues to 100 nucleotide residues, most preferably from 20 to 44 nucleotide residues.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

### DETAILED DESCRIPTION OF THE INVENTION

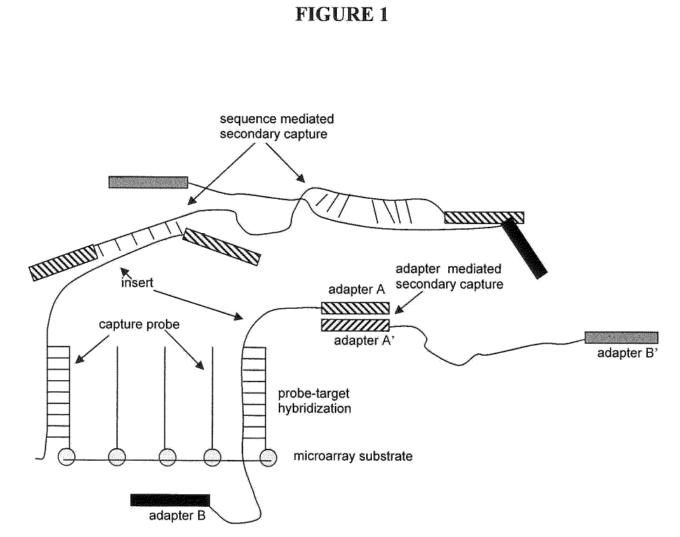

Secondary capture in microarray assays comprises the hybridization based interaction of sequences not represented in the microarray probe capture design (e.g., Alu, THE-1, LINE-1 repeats, etc.) (FIG. 1). One type of secondary capture, for example, is found between non-hybridized sample DNA and the target DNA that is hybridized to a probe (“sequence mediated secondary capture”). Another type of secondary capture is hybridization between adapter or linker sequences that are affixed to the target DNA (“adapter mediated secondary capture”). Adapter mediated secondary capture occurs, for example, when an adapter and its complementary sequence hybridize during a microarray assay. For example, in secondary capture a probe specifically hybridizes to its target, but that target has some non-probe sequences (e.g., Nu, THE-1, LINE-1 repeats, etc.) that also hybridize to non-cis copies. One consequence of secondary capture is the enrichment of specific subsets of repeat elements within a target sample (e.g., non-target sequences), leading to poor overall enrichment of the target region. In essence, the desired target sequence to be enriched by capture on the microarray is swamped out by the co-enrichment of unwanted types of local sequence repeats.

As described herein, methods, systems and compositions of the present invention provide for the suppression of secondary capture in microarray assays thereby increasing the capture of target sequences. Certain illustrative embodiments of the invention are described below. The present invention is not limited to these embodiments.

Competitive, or suppression, hybridization to block secondary capture involves blocking the capture of a potentially strong repetitive DNA signal which can be obtained when using a complex DNA. For example, the DNA is denatured and allowed to re-anneal in the presence of total genomic DNA in solution, or preferably a fraction that is enriched for highly repetitive DNA sequences. In either case, the highly repetitive DNA within the target DNA is present in large excess over the repetitive elements in the probe (since the arrays are most often produced with as little repeat as possible). As a result, such sequences will readily associate with complementary strands of the repetitive sequences within the target, adding massive excess of exogenous copies of the same types of repeats thereby effectively blocking their hybridization to target sequences. As such, blocking agents are used during hybridization reactions.

Instead of using total genomic DNA as a blocking agent in hybridization, it is more effective to use a fraction of total genomic DNA that is enriched for highly repetitive DNA sequence such as Alu, LINE-1 and THE-1 repeats. For complex human DNA and other complex genomes, the latter usually involves preparing a fraction of DNA known as C₀t-1 DNA (e.g., a DNA concentration dependent coefficient of renaturation time of 1.0). C₀t-1 DNA is available commercially or can be prepared using established techniques (see Human Molecular Genetics 2, Eds. Strachan and Read, John Wiley & Sons, Inc.). Maize C₀t-1 DNA was prepared as described herein.

During experimentation it was observed that the employment of C₀t-1 DNA in target enrichment experiments improved on-target sequencing results. However, it was observed that, suprisingly, matching C₀t-1 DNA to the target species provided optimal capture performance. Further, it was determined that when adapter ligated target nucleic acids were used and oligonucleotide sequences complementary to those target sequences were additionally added to hybridization reactions on the micorarray, further improvement was seen with respect to on-target capture. The present invention is not limited to a particular mechanism. Indeed, an understanding of the mechanism is not necessary to practice the present invention. Nonetheless, it is contemplated that this effect is mediated not by supression of non-specific binding capacity of the microarrays, but rather through the excess of specific sequences in the C₀t-1 DNA quenching repeat-mediated secondary capture in the sample DNA.

Classically, hybridization-based sequence characterization employed blocking agents to keep the labelled probe from interacting with the nylon or nitrocellulose solid support. In those studies, the agents assessed to suppress this non-specific DNA binding activity were most typically salmon sperm DNA (salmon genome is comprised predominantly of repeats, more so than mouse or human genomes), or mixtures of purified yeast tRNA. The transition from Southern-type analyses to microarray based studies brought with it the use of non-specific blocking. Currently, controversy exists as to whether or not C₀t-1 is necessary in array hybridization as some applications and manufacturer's (e.g., Agilent Technologies, Abbott Laboratories, etc.) recommend C₀t-1 use for comparative genomic hybridization, and others (Roche NimbleGen, Inc., for example) do not.

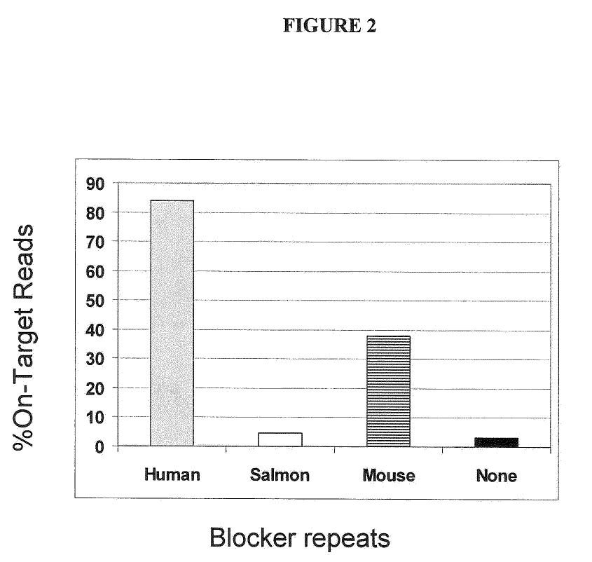

Presently, it is thought that any C₀t-1 DNA is all that is required to block non-specific capture and such blocking results in identical capture performance regardless of the source of blocker. Indeed, currently human C₀t-1 DNA is recommended for use in both human and mouse target sequence capture methods and it is contemplated that using salmon sperm DNA to suppress non-specific hybridizations that might occur during a microarray hybridization assay will work equally well as any C₀t-1 DNA. This is desirable to know as C₀t-1 DNA costs approximately 30× more than salmon-sperm DNA. However, it has been observed that C₀t-1 DNA from different species poisons, for example, a complexity reduction assay where human DNA sequences are the target sequences for capture and enrichment (FIG. 2). In developing embodiments for the present invention, complexity reduction assays were performed in triplicate using the same DNA library pool (either human or mouse) and the same array design. All three captures used the same amount of DNA library (1 ug) and 100 ug of blocker DNA, either C₀t-1 human, C₀t-1 mouse, or salmon sperm DNA. Control captures were performed to help identify success variation. As seen in FIG. 2, human library DNA capture with human C₀t-1 DNA were successful (yielding approximately 85% on-target reads), whereas human assays using mouse C₀t-1 performed badly (considered as failures), and those human assays blocked with salmon sperm DNA were equivalent to assays where no block DNA was present (negative controls, considered as failures). However, it should be noted that higher concentrations of salmon sperm DNA (i.e. 200 micrograms per capture hybridization) gave improved results. A similar pattern is seen when mouse target sequences are used, such that optimal target capture is realized when mouse C₀t-1 is used in lieu of human C₀t-1 or salmon sperm DNA.

To investigate whether this phenomenon was seen when using non-mammalian target nucleic acids, microarray experiments were undertaken wherein DNA from an economically important plant crop species, in this instance Zea mays, was used for target sequence enrichment. For maize target capture a range of potential secondary capture blockers was investigated; salmon sperm DNA, excess maize genomic DNA without adapters, a collection of PCR amplified maize repeat regions, and mung bean nuclease generated maize C₀t-1 DNA. The use of human C₀t-1 DNA in maize target enrichment resulted in approximately 1% (0.94%) on target reads; a failure from a practical perspective.

Quantitative PCR (qPCR) data, as seen in Table 1, demonstrates that exclusion of non-target sequences is optimal and enrichment of target sequences is optimal when maize C₀t-1 DNA is used as the blocking DNA against secondary capture.

TABLE 1

| | | Fold depleted | | Fold enriched | | | | Enrichment | Exclusion | | |
|---|---|---|---|---|---|---|---|---|---|---|---|
| Sample | | GAPC | Actin | Mez1a | Mez1b | Fie1a | Fie1b | Average | Average | Total | Rank |
| B73 first cap | #4 (LMPCR B73) | 367 | ND | 116 | 108 | 181 | 123 | 132 | −367 | −235 | 3 |
| Mo17 first cap | #6 (LMPCR Mo17) | 15 | 18 | 159 | 166 | 242 | 214 | 195 | −16 | 178 | 4 |
| Recent cap1 | B73 cap. 1 no block | 2 | 0 | 1 | ND | 0 | 0 | 0 | −1 | −1 | 6 |
| Recent cap2 | B73 cap. 2 cot1 | 25000 | 25000 | 136 | 58 | 116 | 90 | 100 | 25000 | 24900 | 1 |
| Recent cap3 | B73 cap. 3 PCR block | 594 | 22691 | 156 | 57 | 160 | 112 | 121 | −11642 | −11521 | 2 |
| Recent cap4 | B73 cap. 4 cold B73 | 299 | 2355 | 127 | 30 | 133 | 74 | 91 | −1327 | −1236 | 5 |

Two inbred Zea mays cultivars, B73 and Mo17, were utilized in enrichment microarray assays. The B73 enrichment experiment incorporating salmon sperm DNA into the experiment (B73 cap. 1 no block), demonstrates no enrichment of Mez and Fie target sequences. Use of maize C₀t-1 DNA as blocker (B73 cap.2 cot1) demonstrates maximal depletion of unwanted targets (as followed by qPCR of GAPC and actin) and maximal enrichment of B73 target sequences (Rank 1). Use of 8 PCR amplified maize repetitive sequences was also effective in blocking enrichment of non-target B73 sequences (B73 cap. 3 PCR block (Lamb et al., Chromosome Res. 2007; 15:33-49)), albiet not as well as the maize C₀t-1 DNA. Use of maize C₀t-1 to block secondary capture of non-target sequences allowed on target percentage of target sequences captured to increase approximately 30-36%, depending upon how the analysis was performed. Moreover, when total genomic maize DNA was used as blocker (B73 cap.4 cold B73) target sequences were poorly enriched, thereby suggesting that target sequence molecules can be competed off of the array. As such, one embodiment of the present invention provides species specific blocker consisting mainly of repeated sequences.

When considered together, these data suggest that on target enrichment in complexity reduction assays is a consequence of suppression of repeat-repeat mediated inter-molecular interactions. Even though it is unclear if these interactions are occurring on the solid-support or in solution, it is clear that since salmon sperm was demonstrated to not block secondary capture, it is contemplated that the role of the C₀t-1 is not suppression of non-specific DNA binding capacity of the array. However, the present invention is not limited by the mode of operation or location where blocking of secondary capture of the species specific C₀t-1 DNA occurs.

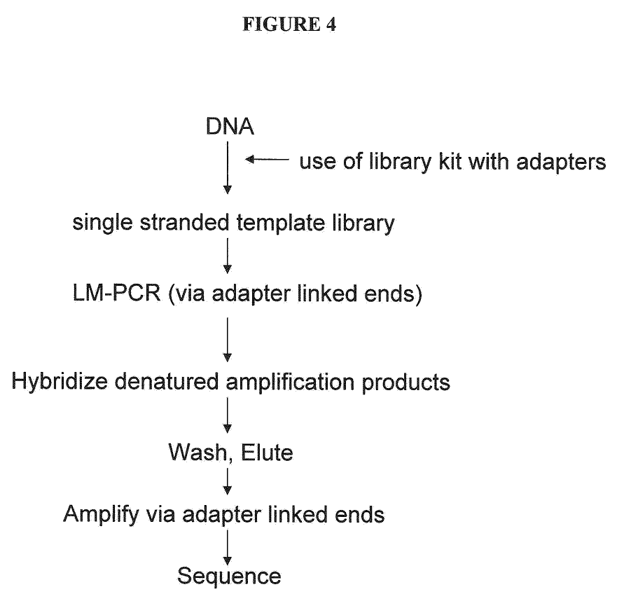

In developing embodiments of the present invention, the effect of longer LM-PCR linkers or adapters (44 bp in lieu of 22-24 bp linkers) affixed to the fragmented target sequences for target sequence enrichment in microarray assays was also investigated. Incorporating LM-PCR adapters onto the ends of target, fragmented DNA allows for, for example, the amplification of genomic DNA prior to the enrichment, with enrichment of target sequences occurring from the amplified population. One exemplary method for adapter attachment is by making a sequencing library, for example, by using a library protocol wherein the enriched targets can be sequenced directly in a sequence analysis protocol from 454 Life Sciences (Branford, Conn.) using a GS FLX sequencer. However, the present invention is not limited by the method used for library generation and sequencing and the present example demonstrates only one possible embodiment of the present invention (e.g., a skilled artisan will recognize alternative methods equally amenable for use with the present invention). An exemplary workflow for LM-PCR adapted target sequences is found in FIG. 4. In some embodiments, secondary capture blocker DNA added to the complexity reduction assays included not only species specific C₀t-1 DNA, but also either short (24 bp) adapter complementary oligonucleotides or longer (44 bp) adapter complementary oligonucleotides (e.g., hybridization blocking oligonucleotides). It is contemplated that the hybridization blocking oligonucleotides are optimal when they reflect a majority of the sequences of the adapter oligonucleotide ligated to the target sample.

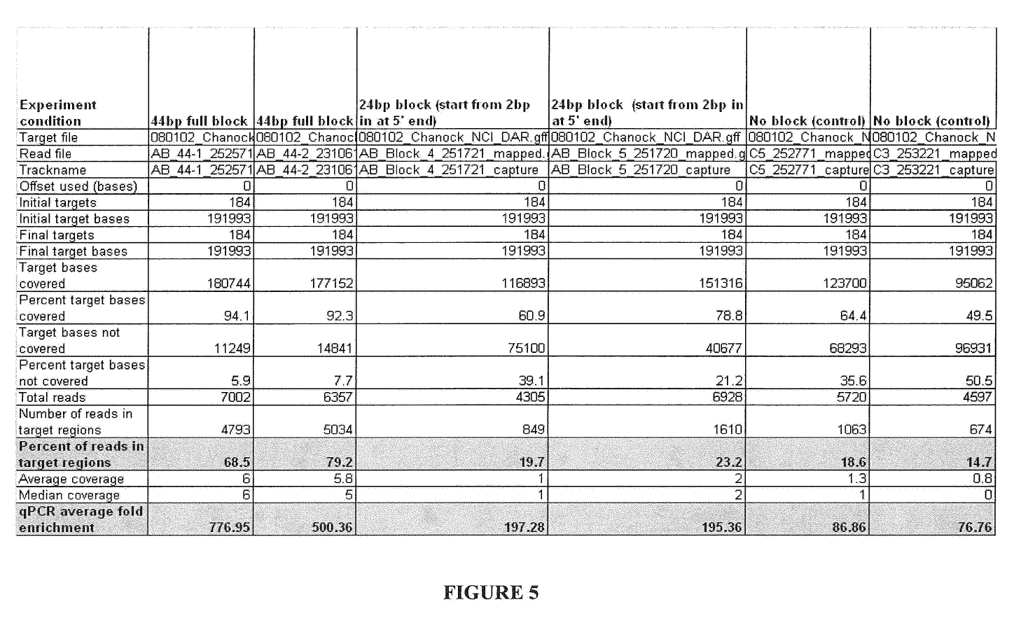

It was demonstrated in replicate parallel experiments that enrichment of the original target sequence libraries was poor with no block or with partial block (24 bp block) in the presence of species specific C₀t-1 DNA resulting in approximately 20% or less on target read capture rate (FIG. 5). However, when oligonucleotides complementary to the 44 bp linkers (e.g., 44 bp full block hybridization oligonucleotide blockers) were added to the hybridization in conjunction with C₀t-1, the capture performance increased up to approximately 70-80% on target captured sequences (percent of reads in target regions). The present invention is not limited to a particular mechanism. Indeed, an understanding of the mechanism is not necessary to practice the present invention. Nonetheless, it is contemplated that the use of C₀t-1 DNA as a blocker suppresses genomic sample sequence-mediated secondary capture, whereas the excess hybridization blocking oligonucleotides suppresses adapter-mediated secondary hybridization complexes. It is contemplated that a 44 bp adapter molecule forms a hybridization complex with at least half of the molecules in the library (e.g., 5′ most 44 bp and 3′ most 44 bp are the same on every molecule). Basically, every strand has the opportunity to hybridize to its complementary strand such that at least 88 total by is double stranded. It is contemplated that blocking oligonucleotides suppress the formation of these inter-molecular complexes (regardless of whether they are on the array or in solution). Further, creating adapter blocking oligonucleotides having non-extendable (e.g., 2′3′ dideoxy) 3′ ends provides for non-interference of the blocking oligonucleotides in LM-PCR post-capture methods (e.g., amplification or sequencing) if any of the blocking oligonucleotides inadvertantly remain after washing of the substrate.

In some embodiments of the present invention, a sample containing denatured (e.g., single-stranded) nucleic acid molecules, preferably genomic nucleic acid molecules, which can be fragmented molecules, is exposed under hybridizing conditions to a plurality of oligonucleotide probes on a microarray substrate. During the hybridization reaction the present invention provides for inclusion of blocking DNA, specifically species specific blocking DNA, wherein said species specific blocking DNA increases the on-target enrichment (e.g., resulting in increases in on-target reads) of target nucleic acids compared to the use of non-species specific blocking DNA during hybridization.

In some embodiments of the present invention, a sample containing nucleic acid molecules, preferably genomic nucleic acid molecules, which can be fragmented molecules, are further modified to comprise adapter linker sequences on both the 5′ and 3′ ends of the fragmented DNA. The adapter sequences can either be self-complementary, non-complementary, or Y type adapters. The adapter sequences are utilized, for example, for ligation mediated amplification of the fragmented nucleic acids as well as for sequencing purposes. Adapter linked fragments are preferentially amplified via LM-PCR and are exposed under hybridizing conditions to a plurality of oligonucleotide probes on a microarray substrate. During the hybridization reaction the present invention provides for inclusion of blocking DNA, specifically species specific blocking DNA, to suppress secondary hybridization reactions wherein said species specific blocking DNA increases the on-target enrichment of target nucleic acids compared to the use of non-species specific blocking DNA during hybridization. In some embodiments, hybridization blocker oligonucleotides are additionally included during hybridization to suppress secondary capture reactions. It is contemplated that the inclusion of species specific blocking DNA in conjunction with hybridization blocker oligonucleotides further increases the enrichment of on-target enrichment compared to microarray hybridizations without such blocking sequences.

It is contemplated that the present invention is not limited by the kind of microarray assay being performed, and indeed any assay where suppression of secondary capture reactions are desired will benefit from practicing the methods and systems of the present invention. Assays include, but are not limited to, complexity reduction and sequence enrichment, comparative genomic hybridization, comparative genomic sequencing, expression, chromatin immunoprecipitation-chip (ChIP-chip), epigenetic, and the like.

In embodiments of the present invention, probes for capture of target nucleic acids are immobilized on a substrate by a variety of methods. In one embodiment, probes can be spotted onto slides (e.g., U.S. Pat. Nos. 6,375,903 and 5,143,854). In preferred embodiments, probes are synthesized in situ on a substrate by using maskless array synthesizers (MAS) as described in U.S. Pat. Nos. 6,375,903, 7,037,659, 7,083,975, 7,157,229 that allows for the in situ synthesis of oligonucleotide sequences directly on a slide.

In some embodiments, a solid support is a population of beads or particles. The beads may be packed, for example, into a column so that a target sample is loaded and passed through the column and hybridization of probe/target sample and blocking nucleic acids (e.g., species specific C₀t-1 DNA and/or hybridization blocking oligonucleotides) takes place in the column, followed by washing and elution of target sample sequences for reducing genetic complexity and enhancing target capture. In some embodiments, in order to enhance hybridization kinetics, hybridization takes place in an aqueous solution comprising multiple probes in suspension in an aqueous environment.

In embodiments of the present invention, the hybridization probes for use in microarray capture methods as described herein are printed or deposited on a solid support such as a microarray slide, chip, microwell, column, tube, beads or particles. The substrates may be, for example, glass, metal, ceramic, polymeric beads, etc. In preferred embodiments, the solid support is a microarray slide, wherein the probes are synthesized on the microarray slide using a maskless array synthesizer. The lengths of the multiple oligonucleotide probes may vary and are dependent on the experimental design and limited only by the possibility to synthesize such probes. In preferred embodiments, the average length of the population of multiple probes is about 20 to about 100 nucleotides, preferably about 40 to about 85 nucleotides, in particular about 45 to about 75 nucleotides. In embodiments of the present invention, hybridization probes correspond in sequence to at least one region of a genome and can be provided on a solid support in parallel using, for example, maskless array synthesis (MAS) technology.

In embodiments of the present invention, the suppression of secondary capture by practicing methods of the present invention is not limited to suppression on a particular substrate, for example suppression of secondary capture of probe and non-target sequences wherein the probes are affixed to the substrate. Indeed, secondary capture that occurs in solution is also suppressed by inclusion of the species specific blocking DNA and/or blocking oligonucleotides as described herein.

The present invention is not limited to the type of sample for capture, and indeed it is contemplated that any sample used is equally applicable to the present invention including, but not limited to, genomic DNA or RNA sample, cDNA library or mRNA library. In some embodiments, nucleic acid sequences used herein are fragmented, wherein said fragments have an average size of about 100 to about 1000 nucleotide residues, preferably about 250 to about 800 nucleotide residues and most preferably about 400 to about 600 nucleotide residues

In embodiments of the present invention, target nucleic acids are typically deoxyribonucleic acids or ribonucleic acids, and include products synthesized in vitro by converting one nucleic acid molecule type (e.g., DNA, RNA and cDNA) to another as well as synthetic molecules containing nucleotide analogues. Fragmented genomic DNA molecules are in particular molecules that arc shorter than naturally occurring genomic nucleic acid molecules. A skilled person can produce molecules of random- or non-random size from larger molecules by chemical, physical or enzymatic fragmentation or cleavage using well known protocols. For example, chemical fragmentation can employ ferrous metals (e.g., Fe-EDTA). Physical methods can include sonication, hydrodynamic force or nebulization (e.g., see European patent application EP 0 552 290). Enzymatic protocols can employ nucleases and partial digestion reactions such as micrococcal nuclease (e.g., Mnase) or exo-nucleases (e.g. Exo1 or Bal31) or restriction endonucleases.

The population of nucleic acid molecules which may comprise the target nucleic acid sequences preferably contains the whole genome or at least one chromosome of an organism or at least one nucleic acid molecule with at least about 100 kb. In particular, the size(s) of the nucleic acid molecule(s) is/are at least about 200 kb, at least about 500 kb, at least about 1 Mb, at least about 2 Mb or at least about 5 Mb, especially a size between about 100 kb and about 5 Mb, between about 200 kb and about 5 Mb, between about 500 kb and about 5 Mb, between about 1 Mb and about 2 Mb or between about 2 Mb and about 5 Mb. In some embodiments, the nucleic acid molecules are genomic DNA, while in other embodiments the nucleic acid molecules are cDNA, or RNA species (e.g., tRNA, mRNA, miRNA).

In embodiments of the present invention, the nucleic acid molecules which may or may not comprise the target nucleic acid sequences may be selected from an animal, a plant or a microorganism. In some embodiments, if limited samples of nucleic acid molecules are available the nucleic acids are amplified (e.g., by whole genome amplification) prior to practicing the method of the present invention. For example, prior amplification may be necessary for performing embodiments of the present invention for forensic purposes (e.g., in forensic medicine, etc.).

In some embodiments, the population of nucleic acid molecules is a population of genomic DNA molecules. The hybridization probes and subsequent amplicons may comprise one or more sequences that target one or more (e.g., a plurality) of exons, introns or regulatory sequences from one or more (e.g., a plurality of) genetic loci, the complete sequence of at least one single genetic locus, said locus having a size of at least 100 kb, preferably at least 1 Mb, or at least one of the sizes as specified above, sites known to contain SNPs, or sequences that define an array, in particular a tiling array, designed to capture the complete sequence of at least one complete chromosome. In some embodiments, only one hybridization probe sequence is utilized to capture a target sequence. Indeed, the present invention is not limited to the number of different probe sequences utilized to capture a target nucleic acid.

It is contemplated that target nucleic acid sequences are enriched from one or more samples that include nucleic acids from any source, in purified or unpurified form. The source need not contain a complete complement of genomic nucleic acid molecules from an organism. The sample, preferably from a biological source, includes, but is not limited to, isolates from individual patients, tissue samples, or cell culture. The target region can be one or more continuous blocks of several megabases, or several smaller contiguous or discontiguous regions, such as all of the exons from one or more chromosomes, or sites known to contain SNPs. For example, the one or more hybridization probes comprising one, or multiple different, sequence(s) and subsequent probe derived amplicons can support an array (e.g., non-tiling or tiling) designed to capture one or more complete chromosomes, parts of one or more chromosomes, one exon, all exons, all exons from one or more chromosomes, selected one or more exons, introns and exons for one or more genes, gene regulatory regions, and so on.

Alternatively, to increase the likelihood that desired non-unique or difficult-to-capture targets are enriched, the probes can be directed to sequences associated with (e.g., on the same fragment as, but separate from) the actual target sequence, in which case genomic fragments containing both the desired target and associated sequences will be captured and enriched. The associated sequences can be adjacent or spaced apart from the target sequences, but a skilled person will appreciate that the closer the two portions are to one another, the more likely it will be that genomic fragments will contain both portions.

In some embodiments of the present invention, the methods comprise the step of ligating adapter or linker molecules to one or both ends of fragmented nucleic acid molecules prior to denaturation and hybridization to the probes. In some embodiments of the present invention the methods further comprise amplifying said adapter modified nucleic acid molecules with at least one primer, said primer comprising a sequence which specifically hybridizes to the sequence of said adapter molecule(s). In some embodiments of the present invention, double-stranded adapters are provided at one or both ends of the fragmented nucleic acid molecules before sample denaturation and hybridization to the probes. In such embodiments, target nucleic acid molecules are amplified after elution to produce a pool of amplified products having further reduced complexity relative to the original sample. The target nucleic acid molecules can be amplified using, for example, non-specific Ligation Mediated-PCR (LM-PCR) through multiple rounds of amplification and the products can be further enriched, if required, by one or more rounds of selection against the microarray probes. The linkers or adapters are provided, for example, in an arbitrary size and with an arbitrary nucleic acid sequence according to what is desired for downstream analytical applications subsequent to the complexity reduction step. The adapter linkers can range between about 12 and about 100 base pairs, including a range between about 18 and 100 base pairs, and preferably between about 20 and 44 base pairs. In some embodiments, the linkers are self-complementary, non-complementary, or Y adapters.

Ligation of adapter molecules allows for a step of subsequent amplification of the captured molecules. Independent from whether ligation takes place prior to or after the capturing step, there exist several alternative embodiments. In one embodiment, one type of adapter molecule (e.g., adapter molecule A) is ligated that results in a population of fragments with identical terminal sequences at both ends of the fragment. As a consequence, it is sufficient to use only one primer in a potential subsequent amplification step. In an alternative embodiment, two types of adapter molecules A and B are used. This results in a population of enriched molecules composed of three different types: (i) fragments having one adapter (A) at one end and another adapter (B) at the other end, (ii) fragments having adapters A at both ends, and (iii) fragments having adapters B at both ends. The generation of enriched molecules with adapters is of outstanding advantage, if amplification and sequencing is to be performed, for example using the 454 Life Sciences Corporation GS20 and GS FLX instrument (e.g., see GS20 Library Prep Manual, December 2006, WO 2004/070007; incorporated herein by reference in their entireties).

In embodiments of the present invention, methods, systems and compositions comprise the incorporation of species specific blocking DNA into a hybridization reaction in a microarray assay for suppression of secondary capture. In some embodiments, the species specific blocking DNA is C₀t-1 DNA from any species including, but not limited to, mammalian species, plant species, non-mammalian species, bacterial species, yeast species, etc. In some embodiments of the present invention, a microarray hybridization reaction wherein the target nucleic acid sequences for hybridization are human nucleic acid sequences comprises human C₀t-1. In some embodiments of the present invention, a microarray wherein the target nucleic acid sequences are murine nucleic acid sequences comprises murine C₀t-1. In some embodiments of the present invention, a microarray wherein the target nucleic acid sequences are plant nucleic acid sequences comprises plant C₀t-1. It is contemplated that the present invention is not limited to any particular plant species. Examples of plant species utilized with the present invention include, but are not limited to, economically and/or research relevant plant species such as corn, soybean, sorghum, wheat, vegetable crops, fruit crops, forage crops, grasses, broadleaf plants and any other dicot and/or monocot plants.

In embodiments of the present invention, methods, systems and compositions comprise the incorporation of hybridization blocking oligonucleotides into a hybridization reaction in a microarray assay for suppression of adapter mediated secondary capture. In some embodiments, the hybridization blocking oligonucleotides is used in conjunction with species specific C₀t-1 DNA from any species including, but not limited to, mammalian species, plant species, non-mammalian species, bacterial species, yeast species, etc. In some embodiments, the sequence of the hybridization blocking oligonucleotides is derived from the sequence of the one or more adapter molecules ligated to fragmented nucleic acids. In some embodiments, the sequence of the hybridization blocking oligonucleotides comprises the whole sequence of the one or more adapter molecules, whereas in other embodiments the sequence of the hybridization blocking oligonucleotides comprises a fragment of the sequence of the one or more adapter molecules. In some embodiments of the present invention, a microarray hybridization reaction wherein the target nucleic acid sequences for hybridization are human nucleic acid sequences comprises human C₀t-1 in conjunction with hybridization blocking oligonucleotides. In some embodiments of the present invention, a microarray wherein the target nucleic acid sequences are murine nucleic acid sequences comprises murine C₀t-1 in conjunction with hybridization blocking oligonucleotides. In some embodiments of the present invention, a microarray wherein the target nucleic acid sequences are plant nucleic acid sequences comprises plant C₀t-1 in conjunction with hybridization blocking oligonucleotides. It is contemplated that the present invention is not limited to any particular plant species. Examples of plant species utilized with the present invention include, but are not limited to, economically and/or research relevant plant species such as corn, soybean, sorghum, wheat, vegetable crops, fruit crops, forage crops, grasses, broadleaf plants and any other dicot and/or monocot plants.

In some embodiments, the present invention comprises a kit comprising reagents and materials for performing methods according to the present invention. Such a kit may include one or more microarray substrates upon which is immobilized a plurality of hybridization probes specific to one or more target nucleic acid sequences from one or more target genetic loci (e.g., specific to exons, introns, SNP sequences, etc.), a plurality of probes that define a tiling array designed to capture the complete sequence of at least one complete chromosome, amplification primers, reagents for performing polymerase chain reaction methods (e.g., salt solutions, polymerases, dNTPs, amplification buffers, etc.), reagents for performing ligation reactions (e.g., ligation adapters, T14 polynucleotide kinase, T4 DNA ligase, buffers, etc.), species specific blocking DNA, hybridization blocking oligonucleotides, tubes, hybridization solutions, wash solutions, elution solutions, magnet(s), and tube holders. In some embodiments, a kit further comprises two or more different double stranded adapter molecules.

In some embodiments, a kit further comprises at least one or more compounds from a group consisting of DNA polymerase, T4 polynucleotide kinase, T4 DNA ligase, one or more array hybridization solutions, and/or one or more array wash solutions. In preferred embodiments, three wash solutions are included in a kit of the present invention, the wash solutions comprising SSC, DTT and optionally SDS. For example, kits of the present invention comprise Wash Buffer I (0.2% SSC, 0.2% (v/v) SDS, 0.1 mM DTT), Wash Buffer II (0.2% SSC, 0.1 mM, DTT) and/or Wash Buffer III (0.05% SSC, 0.1 mM DTT). In some embodiments, systems of the present invention further comprise an elution solution, for example water or a solution containing TRIS buffer and/or EDTA.

### EXPERIMENTAL

The following examples are provided in order to demonstrate and further illustrate certain preferred embodiments and aspects of the present invention and are not to be construed as limiting the scope thereof.

### Example 1

### Enrichment and Sequencing of Human and Mouse DNA

Experiments where mouse and/or human samples were utilized followed established protocols for microarray analysis using C₀t-1 DNA to block secondary hybridization. Examples of the protocols and methods used herein are found in NimbleGen Arrays User's Guide; Sequence Capture Array Delivery (Roche NimbleGen, Inc.) and 454 BioSciences GS GLX Shotgun DNA Library Preparation Method Manual (454 BioSciences), both of which are incorporated herein by reference in their entireties.

### Example 2

### Generation of Maize C₀t-1

Three hot block dry baths were set at 105° C., 65° C. and 37° C. Three hundred micrograms of maize DNA was sonicated in water in a total volume of 420 ul in a 2 ml Dolphin nose tube. Probe sonication was performed for a total of 3 times. After sonication, 30 ul of 5M NaCl and 50 ul water was added to bring the total volume to 500 ul (0.3M NaCl) to yield approximately 1 ng/ul DNA concentration.

The diluted sample tubes were heated in the hottest heat block for 10 min., followed by a quick spin tube and incubation at 65° C. for 19 min., followed by the addition of 60 ul of 10× Mung Bean Nuclease buffer (Promega Corporation, Madison Wis.) and 36.6 ul room temperature water. One unit (1U) of Mung Bean Nuclease was added per microgram of DNA. The reactions were vortexed, spun down, and incubated at 37° C. for 10 min. Following incubation, 1.2 ml of Zymo DNA binding buffer (Zymo Research) was added, the tubes were vortexed, the DNA solution dispensed (150 ul) into each of 12 Zymo 25 ug clean-up columns and the columns were washed. DNA was eluted from columns in 35 ul water. DNA sample purity was verified by 260/280 ratio (at least 1.7) with a sample aliquot further assessed by agarose gel electrophoresis to check for degradation.

### Example 3

### Adapter Ligated Human DNA Library and Assays

Adapter oligonucleotides were created and ligated to the ends of fragmented human DNA. Adapter oligonucleotides, A, A′, B and B′, were synthesized and resuspended in Tris-EDTA buffer to a concentration of 400 μM (*-Phosphorothioate Bond, /5BioTEG/-5′ Biotin-TEG).

Adapter A (SEQ ID NO: 1):
C*C*A*T*CTCATCCCTGCGTGTCCCATCTGTTCCCTCCCTGTC*T*C*A
*G
Adapter A′ (SEQ ID NO: 2):
C*T*G*A*GACA*G*G*G*A
Adapter B:

(SEQ ID NO: 3)

/5BioTEG/C*C*T* A*TCCCCTGTGTGCCTTGCCTATCCCCTGTTGCG
TGTC*T*C* A*G
Adapter B′ (SEQ ID NO: 4):
C*T*G*A*GACA*C*G*C*A

Oligonucleotides A&A′ and B&B′(5 μl of 400 μM linker solution) were aliquoted into two separate PCR tubes and 90 μl of annealing buffer (250 μl of 1M MgOAc in 500 μl 1M Tris-HCl (pH 7.8) in total volume of 50 ml water) was added to create 20 μM solutions of the oligonucleotides adapters (A&A′, B&B′). Equal amounts of 20 μM adapter solutions were mixed together and allowed to anneal using the following program; 95° C. for 1 min, ramping temperature at 0.1° C./sec. to 15° C., lower temperature to 4° C., store at −20° C.

The genomic DNA sample was fragmented using a nebulizer. The DNA sample (5 μg) in a total volume of 100 μl TE was combined with 500 μl of ice cold nebulization buffer (53.1% glycerol, 37 mM Tris-HCl, 55 mM EDTA (pH 7.5) in water). A non-reactive gas was passed through the DNA solution (at 45 psi) for 1 min. Two milliliters of PBI buffer (Qiagen) was added to the nebulized sample, the sample was split and applied to Qiagen MinElute columns (2), eluted with 24 μl Elution buffer (EB, Qiagen), and the eluted samples recombined with final volume adjusted to 50 μl.

Small fragments (approximately 250 bp or less) were removed from the nebulized sample by the addition of 35 μl of AMPure® beads (Agencourt® Bioscience Corporation), incubation for 5 min. at room temperature, washing beads with ethanol (twice with 500 μl), drying beads at 37° C. for approximately 4 min, and eluting the bead captured fragmented sample DNA with addition of 25 μl EB. The ends of the captured, fragmented DNA were polished using T4 Polynucleotide Kinase and T4 DNA polymerase using established protocols (Molecular Cloning; A Laboratory Manual, Eds. Sambrook et al., Cold Spring Harbor Press).

The adapter complexes were ligated to the polished DNA fragments. Fifty microliters of the polished DNA were added to a Quick Ligation™ reaction (New England BioLabs, Inc.) of 60 μl 2× Quick ligation buffer, 5 μl of 10 μM adapter solution and 5 μl Quick ligase. The ligation reaction was incubated at 25° C. for 15 min. Following ligation, the adapter linked sample DNA fragments were purified away from reaction components by adding 600 μl of PBI, applying the solution to a MinElute column (Qiagen), centrifuging 1 min. (16K×g), adding 750 μl PE buffer (Qiagen), centrifuging for 1½ min. (16K×g), drying the column by centrifuging for an additional 1 min., adding 26 μl EB and incubating the column for 1 min. at room temperature and eluting the purified adapter linked DNA library fragments from the column by final centrifugation for 1 min (16K×g).

Ligation mediated PCR (LM-PCR) was performed on the DNA adapter linked library. Briefly, Pfu DNA polymerase was utilized for amplifying the fragments using primers specific to linker sequences:

| | |
|---|---|
| Primer A | |
| 5′ATCTCATCCCTGCGTGTCCCATCT 3′ | (SEQ ID NO: 5) |
| Primer B | |
| 5′TATCCCCTGTGTGCCTTGCCTATC 3′ | (SEQ ID NO: 6) |

Polymerase chain reaction was performed; 95° C. 2 min., 12 cycles of 95° C. for 30 sec., 63.5° C. for 30 sec., 72° C. for 1 min., with a final extension at 72° C. for 1 min. and storage at 4° C. The amplification products were purified using QIAquick columns as previously described. Sample concentration and amplification size range were determined. Sample range is typically between 300-1000 bp with A260/280 ratio between 1.7-2.0.

The amplified samples were hybridized to microarrays using hybridization system and reagents from Roche NimbleGen, Inc. following manufacturer's protocols, for example those found in the NimbleGen Arrays User's Guide; Sequence Capture Array Delivery (Roche NimbleGen, Inc.), incorporated herein by reference in its entirety. Briefly, 100 μl of human C₀t-1 DNA was added to 5 μg adapter linked fragmented human DNA. Alternatively, in some experiments the C₀t-1 DNA was supplemented by the addition of experimental hybridization blocking oligonucleotides of either 44 bp or 24 bp in length (/ddC/-cytidine 2′3′ dideoxyribonucleotide):

Hybridization blocking oligo A44 (SEQ ID NO: 7):
CCATCTCATCCCTGCGTGTCCCATCTGTTCCCTCCCTGTCTCAG/ddC/
Hybridization blocking oligo B44 (SEQ ID NO: 8):
CCTATCCCCTGTGTGCCTTGCCTATCCCCTGTTGCGTGTCTCAG/ddC/
Hybridization blocking oligo A24 (SEQ ID NO: 9):
ATCTCATCCCTGCGTGTCCCATCT/ddC/
Hybridization blocking oligo B24 (SEQ ID NO: 10):
TATCCCCTGTGTGCCTTGCCTATC/ddC/

Samples were dried down, rehydrated, denatured, applied to the microarray and hybridized at 42° C. for approximately 48 hours. Washing and elution of the enriched human DNA was performed.

The enriched sample DNA was subjected to post-capture LM-PCR as previously described except that the amplification cycling was performed 24 times. Amplified samples were purified using QIAquick columns as previously described.

Enriched, linker adapted human library DNA samples were prepared for sequencing and sequenced on a 454 BioSciences GS FLX System. Methods for sample preparation for sequencing are found in, for example, the 454 BioSciences GS GLX Shotgun DNA Library Preparation Method Manual, incorporated herein by reference in its entirety.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method of suppressing secondary capture in a nucleic acid hybridization comprising the steps of:
a) immobilizing one or more nucleic acid probes to capture target nucleic acid sequences in a sample,
b) adding a secondary capture suppressing agent to said sample wherein said secondary capture suppressing agent comprises a target nucleic acid species specific C₀t-1 DNA and a hybridization blocking oligonucleotide, and
c) applying said sample and said secondary capture suppressing agent to said probes for hybridization to said target sequences.

2. The method of claim 1, wherein said species specific C₀t-1 DNA is human C₀t-1 DNA and said target nucleic acid sequences are human nucleic acids.

3. The method of claim 1, wherein said species specific C₀t-1 DNA is mouse C₀t-1 DNA and said target nucleic acid sequences are mouse nucleic acids.

4. The method of claim 1, wherein said species specific C₀t-1 DNA is plant C₀t-1 DNA and said target nucleic acid sequences are plant nucleic acids.

5. The method of claim 4 wherein said plant C₀t-1 is maize C₀t-1 and said plant nucleic acids are maize nucleic acids.

6. The method of claim 1, wherein said secondary capture suppressing agent is present in a molar excess of at least 5 fold relative to the target nucleic acid sequences.

7. The method of claim 1, wherein said target nucleic acid sequences comprise a DNA library and wherein said library comprises self-complementary adapters.

8. The method of claim 1, wherein said target nucleic acid sequences comprise a DNA library and wherein said library comprises non-complementary adapters.

9. The method of claim 1, wherein said target nucleic acid sequences comprise a DNA library and wherein said library comprises Y based adapters.

10. The method of claim 1, wherein said hybridization blocking oligonucleotide has a sequence which is complementary to adapter ligated nucleic acids.

11. A method of suppressing secondary capture in a nucleic acid hybridization comprising the steps of:
a) immobilizing one or more nucleic acid probes to capture target nucleic acid sequences in a sample,
b) adding a secondary capture suppressing agent to said sample wherein said secondary capture suppressing agent comprises a hybridization blocking oligonucleotide which is complementary to adapter ligated nucleic acids, and
c) applying said sample and said secondary capture suppressing agent to said probes for hybridization to said target sequences.

12. A composition comprising species specific C₀t-1 DNA and hybridization blocking oligonucleotides wherein said composition is used in blocking secondary capture in hybridization based assays.

13. The composition of claim 12, wherein said species specific. C₀t-1 DNA is from the group consisting of human C₀t-1, mouse C₀t-1 or plant C₀t-1.

14. The composition of claim 12, wherein said hybridization blocking oligonucleotides have sequences which are complementary to adapter ligated nucleic acids.

15. The composition of claim 13, wherein said plant C₀t-1 is maize C₀t-1.
